(19) United States
(12) Patent Application Publication
(10) Pub. No.: US 2026/0120802 A1
(43) Pub. Date: Apr. 30, 2026

(54) ALLELE-SPECIFIC COPY NUMBER DETECTION FROM LOW COVERAGE GENOTYPING DATA

(71) Applicant: SOPHiA GENETICS S.A., Rolle (CH)

(72) Inventors: Christian Pozzorini, Lausanne (CH); Tommaso Coletta, Lausanne (CH); Zhenyu Xu, Nyon (CH)

(73) Assignee: SOPHiA GENETICS S.A., Rolle (CH)

(21) Appl. No.: 19/003,662

(22) Filed: Dec. 27, 2024

Related U.S. Application Data
(60) Provisional application No. 63/615,258, filed on Dec. 27, 2023.

(51) Int. Cl. G16B 30/10; G16B 20/40; G16B 40/20

(52) U.S. Cl. CPC G16B 30/10; G16B 20/40; G16B 40/20

(57) ABSTRACT

Provided is a computer-implemented method for characterization of a sample from low-coverage genotyping data, the method comprising obtaining a sequencing data from the sample; aligning the sequencing data obtained for the sample to the reference genome to generate a read alignment file; identifying at least one informative variant; for each of the at least one locus containing an informative variant from the read alignment file, computing NAltᵢ, comprising: computing a number of reads supporting the presence of the variant, and computing a depth of sequencing at the locus; modeling, over each of the at least one genomic loci, according to a normalized coverage and an observed variant fraction, an allele-specific copy number for the at least one genomic loci; and outputting, for each of the at least one genomic loci, at least one of an absolute copy number or an allelic composition.

### CROSS-REFERENCE TO RELATED APPLICATIONS

This application claims the benefit of U.S. Provisional Patent Application No. 63/615,258, filed on Dec. 27, 2023, which is incorporated by reference herein in its entirety.

### FIELD OF THE DISCLOSURE

The present disclosure relates to a computer-implemented system and method for estimating allele-specific copy numbers from low-coverage genotyping data.

### INTRODUCTION

Errors that occur during mitosis and meiosis can result in duplications and deletions of chromosomal regions ranging from one base pair (bp), a few kilobases (kb) to several megabases (Mb) and entire chromosomes. In diploid organisms, such events change the frequency of alleles within the individual, removing or adding at least one allele at the affected positions. The gene dosage imbalance caused by events that impact allele copy number, including CNV and loss of heterozygosity (LOH), in regions that contain genes or other functional elements can cause disease, including rare inherited disorders and cancer.

Several approaches have been developed to determine the allele copy number of chromosomal regions. Here, the method focuses on approaches that are suitable for clinical determination of allele copy number, which must be compatible with tools used frequently by individuals who are not bioinformatic experts (for example, clinicians who utilize bioinformatic tools, but do not generally construct, nor potentially understand, the backend of workflows themselves). In this context, methods must be cost-effective to support testing of large numbers of patients and should be easy to deploy in various institutions and research centers. In addition, methods that allow comprehensive detection of allele copy number and can be combined with detection of other disease-causing mutations or biomarkers are preferred.

Amongst the most common methods for allele copy number detection in the clinical setting are multiplex ligation-dependent probe amplification (MLPA), microarray based comparative genomic hybridization (aCGH) and SNP microarrays, fluorescence in situ hybridization (FISH) and PCR-based methods. While commonly used, these methods require extensive expertise and specific equipment, are laboratory intensive and, with the exception of SNP arrays, are all low throughputs. In addition, integration of these detection methods with methods to detect other types of genetic variants is limited by the need for performing separate tests or extending the targeted regions considered. For example, MLPA, which is currently the gold standard for CNV detection in the clinical setting, is relatively low throughput and limited by the number of regions targeted by the solution, with regions not covered by probes remaining undetected. Array-based methods allow analysis of more genomic regions, as numerous probes, each designed to capture a specific genomic region, can be combined. Analyses enabled by array-based methods however remain limited by the set of probes included in the test. Thus, it would be beneficial to provide a method that utilizes the next-generation sequencing (NGS; also referred to as high-throughput sequencing) technology to infer allele-specific frequencies, LOH, and, in some instances, compute tumor ploidy and tumor fraction. The use of NGS technology provides a marked improvement over the array-based methods by increasing the number of potential regions covered and thereby boosting scalability, for example, by covering markers for a wide array of diseases.

NGS based techniques offer a scalable solution to support detection of events that impact allele copy number. NGS-based approaches can support comprehensive analysis of all variant types, including allele copy number changes potentially making molecular screening more complete and time-efficient. However, the use of NGS, in particular whole-genome sequencing, in the clinical setting is still limited by the relatively high sequencing costs. Because of the associated sequencing cost reduction, methods that rely on low sequencing coverage (low pass whole-genome sequencing [lpWGS], e.g. <10× coverage depth) have gained traction in the field. Indeed, these methods have already been shown to allow the detection of changes in the genomic boundaries of the regions impacted by copy number alterations, as well as to estimate the number of copies in the region relative to a baseline and are particularly suitable for detection of CNVs. Ho, S. S., Urban, A. E., & Mills, R. E. (2020), Structural variation in the sequencing era. Nature reviews. Genetics, 21(3), 171-189. doi.org/10.1038/s41576-019-0180-9 and Smolander, J., Khan, S., Singaravelu, K. et al. Evaluation of tools for identifying large copy number variations from ultra-low-coverage whole-genome sequencing data. BMC Genomics 22, 357 (2021), doi.org/10.1186/s12864-021-07686-z. In addition, U.S. Patent Pub. No. 2022/0084626 to Pozzorini et al. teaches methods that use lpWGS that can be easily combined with other NGS-based methods for detection of disease-causing variants and biomarkers, making these approaches particularly relevant for the development of solutions that support comprehensive genomic profiling.

In contrast to determination of the genomic boundaries of the copy number change and the relative copy number change of the region, which is sufficient to characterize most CNV events and that can be estimated using lpWGS, determination of absolute copy number changes of CNVs and other allele copy number changes, including LOH, cannot be determined from standard coverage depth analysis of lpWGS data. This is because the absolute number of DNA copies and allele composition for each of the copies cannot be extracted from analysis of changes in coverage data alone. In addition, the coverage depth of lpWGS data (typically <10× coverage) cannot support accurate variant calling at all sequenced positions.

In addition to establishing the copy number of the region, quantitative determination of the frequency of the two alleles, referred to in the field as B-allele frequency, allows to establish the absolute frequency of alleles present in the sample which is relevant to determine LOH events and establish the absolute copy number of changes, for example to help determine disease-causing or biomarker events.

Determination of absolute allele frequency generally involves performing the following operations:
i) Segmentation of the whole genome into regions with the same copy number;
ii) for a given group of genomic segments with the same copy number, determination of the absolute number of DNA copies in the sample (CN=0, 1, 2, 3, . . . );
iii) inference of the allele composition for each of the copy number levels identified (i.e., CN=3, can be realized in the following ways AAA, AAB, ABB, BBB, where A and B denote the genome reference and the alternative, which is different from the A, allele).

In the case of a sample with DNA from more than one origin, including DNA from related or unrelated individuals or mixes of DNA from different cell origins from the same individual, for example due to mosaicism or tumor DNA, determination of the genome segmentation, absolute copy number, and the inference of the allele composition quantities allows determination of two additional biologically relevant quantities: the sample purity (i.e., the tumor content of the sample in the case of a cancer, or contribution of each cell line in the case of mosaicism), and the ploidy of the different samples (e.g., average copy number in the tumor in the case of a cancer or in each cell line in the case of mosaicism). The estimation of these two biologically relevant quantities is, per se, an important and valuable output of estimation of absolute allele frequency determination.

The above problem has been extensively covered in the literature from different perspectives including the use of different algorithms (e.g., HMM, circular binary segmentation) and different sources of data (e.g., NGS or SNP-array). In brief, determination of genome segmentation to establish which regions of the genome have the same copy number can be done using analysis of differences in intensity of signal supporting the presence of the regions, such as what can be obtained by analyzing sequencing coverage. However, the key to efficiently performing steps ii) and iii) is the determination of the B-allele fraction across the genome. Determination of B-allele using most approaches relies on variant calling and determination of variant fraction across the regions and the majority of NGS-based approaches tackling ii) and iii) rely on the targeted sequencing of large panels such as clinical-exome sequencing (CES) or whole-exome sequencing (WES) and may require the sequencing of a matched normal sample.

A variety of methods using lpWGS are already available to segment and identify regions of the genome that have the same copy number (i.e., point i) above). The copy number of these regions outputted by these methods is relative (for example, region A has two times more or less copies than region B).

To support determination of absolute copy number, particularly in the context of mixed cell populations, Carter et al. (PMID: 22544022) has developed a method where segmented copy-number data for the genome of the sample of interest, together with pre-computed models of recurrent karyotypes and, optionally, allelic fraction values for somatic point mutations, are used to infer absolute allelic copy number profiles in cancer samples. This method relies on knowledge of recurrent karyotypes to establish copy number in the sample.

More recently, WO Patent Pub. No 2017/161175 to Ha et al. discloses ichorCNA. ichorCNA simultaneously predicts segments of somatic copy number alterations and estimates of tumor fraction while accounting for sub-clonality and tumor ploidy from low-pass whole-genome sequencing. ichorCNA uses a probabilistic model, implemented as a hidden Markov model (HMM), to simultaneously segment the genome, predict large-scale copy number alterations, and estimate the tumor fraction through the analysis of data obtained from ultra-low-pass whole-genome sequencing sample (ULP-WGS). See also, Adalsteinsson, V. A., Ha, G., Freeman, S. S. et al. Scalable whole-exome sequencing of cell-free DNA reveals high concordance with metastatic tumors. Nat Commun 8, 1324 (2017). doi.org/10.1038/s41467-017-00965-y. ichorCNA is optimized for ultra-low coverage (˜0.1×) sequencing of samples. To address the challenges of the absence of allele fraction information, a consequence of the use of (ultra) low-pass whole-genome sequencing data, ichorCNA focuses on copy number alterations, inferred using a Bayesian model from coverage information. ichorCNA therefore provides a tool to establish tumor fraction and identify copy number alterations from low-coverage data. The method is however not suitable to establish allele copy numbers and allelic composition (i.e., number of copies of each allele at a given locus), identify LOH, or detect other patterns that involve single-nucleotide variants.

Methods that allow determination of allele copy numbers from lpWGS alone and without prior knowledge of common karyotypes to predict allele copy number and infer purity and ploidy are currently missing.

Genetic variation is non-randomly distributed and tends to be organized into specific combinations of variants, located on a chromosome region. These regions, known as haplotypes, tend to be inherited together. The haplotype organization of genetic variation along the genome has been used in population genetics to input missing genetic information and infer population history and the genetic basis of traits and diseases. See Rubinacci S, Hofmeister R J, Sousa da Mota B, Delaneau O. Imputation of low-coverage sequencing data from 150,119 UK Biobank genomes. Nat Genet. 2023 July; 55(7): 1088-1090. doi: 10.1038/s41588-023-01438-3. Epub 2023 Jun. 29. PMID: 37386250; PMCID: PMC10335927.

The novelty of the present approach consists, in part, in its ability to infer from the IpWGS data the B-allele fraction in samples with mixed cell lines, where in addition to genome copy number, the fraction of the different cell lines in the sample (e.g., sample purity in the case of a cancer) will also influence the number of reads supporting the presence of a given alteration. One of the key aspects of the present approach, that supports its performance at low coverage, is the inference of likely informative variants (hereafter referred to as informative variants), based exclusively on analyses of the sample data and population databases.

One advantage of this approach, which allows allele-specific copy number determination in samples with mixed cell lines, is that it relies on lpWGS alone, therefore reducing the costs required for genome sequencing. Indeed, the cost of a sequencing run, with an approximately constant output in number of bases reads, is determined by the sequencing platform and reagent costs. The cost per sample can be decreased by sequencing simultaneously multiple samples, thereby dividing the platform plus reagent cost among samples. However, the output in number of bases reads is also divided by the number of samples. An assay that requires less bases reads per sample is therefore cheaper for a given sequencing platform and reagent. In addition, an assay requiring lower sequencing coverage results in lower costs of data storage.

The method presented here is therefore cheaper and less resource-intensive relative to methods that rely on the use of several data generation workflows.

Currently, no proposal teaches or suggests inferring the B-allele fraction across the entire genome from lpWGS data. This is because lpWGS does not allow variant calling with confidence due to the very small number of reads that typically map to one position in data sets generated using this method. In one embodiment, the computer implemented method may produce an NGS alternative to SNP-array, high coverage whole-genome sequencing, and whole-exome sequencing.

In a further embodiment of the present disclosure, the method may leverage the B-allele fraction obtained to perform allele-specific copy number calling and purity-ploidy estimation in either germline or somatic samples.

### SUMMARY

The present disclosure may provide for a computer-implemented method for characterization of a sample from low-coverage genotyping data. The method may comprise the steps of obtaining a sequencing data from the sample; aligning the sequencing data obtained for the sample to the reference genome to generate a read alignment file; and identifying at least one informative variant. Further, the method may include the steps of, for each of the at least one locus containing an informative variant from the read alignment file, computing NAltᵢ, comprising computing a number of reads supporting the presence of the variant, and computing a depth of sequencing at the locus. In an embodiment, the method may further comprise the steps of modeling, over each of the at least one genomic loci, according to a normalized coverage and an observed variant fraction, an allele-specific copy number for the at least one genomic loci; and outputting, for each of the at least one genomic loci, at least one of an absolute copy number or an allelic composition.

In an embodiment, the step of identifying the at least one informative variant involves filtering out those that are likely homozygous. In another embodiment, the step of identifying the at least one informative variant further comprises identifying variants that are polymorphic in a population database.

In an embodiment, the population database is selected among multiple possible population databases as the one that most closely matches the sample genotype.

In an embodiment, the step of identifying the at least one informative variant comprises filtering out variants having a minor allele frequency (MAF) below 30% in a reference population. In a further embodiment, the step of identifying the at least one informative variant comprises filtering out variants having a minor allele frequency (MAF) below a threshold in the range of 5% to 40% in a reference population.

In an embodiment, the method comprises modeling the allele-specific copy number comprises estimating with a Hidden Markov Model (HMM) the most likely hidden variant fraction for the at least one genomic locus, wherein the observed states comprise the number of reads supporting the variant and the sequencing depth of the locus. In one embodiment, the hidden states are given different weights based on a binomial distribution, a Beta binomial distribution, a uniform distribution, a normal distribution, a Poisson distribution, or another pre-existing distribution. In one embodiment, the hidden states are given different weights based on the Hardy-Weinberg Equilibrium expected based on the variant frequency in a reference population. In an embodiment, the weights based on Hardy-Weinberg Equilibrium are corrected to account for inbreeding in the population. In a further embodiment, the inbreeding coefficient is optimized based on the sample.

In an embodiment, the method further comprises determining at least one genomic event corresponding to the absolute copy number and the allelic composition.

In an embodiment, the method further comprises identifying loss-of-heterozygosity events.

In an embodiment, the sequencing data is derived from whole-genome sequencing, and wherein the whole-genome sequencing is low-pass whole-genome sequencing.

In an embodiment, the sequencing data is derived from whole-exome sequencing or large panel sequencing, and wherein the sequencing coverage is below 10×.

In an embodiment, the sample represents a germline sample and comprises DNA isolated from blood, saliva, or another tissue.

In an embodiment, the sample comprises cell-free DNA (cfDNA) isolated from a liquid biopsy.

In an embodiment, the sample comprises DNA from a tumor.

In an embodiment, identifying informative variants involves filtering out variants above a given variant allele fraction (VAF) threshold in a matched germline reference sample. In one embodiment, the VAF threshold is between 50% and 99%.

In an embodiment, the sample comprises DNA from more than one origin.

In an embodiment, the outputted results include the sample purity, the sample ploidy, or their combination.

In an embodiment, the estimated sample purity, and the estimated sample ploidy are an estimated tumor sample purity and an estimated tumor sample ploidy, respectively.

Additional aspects related to this disclosure are set forth, in part, in the description which follows, and, in part, will be obvious from the description, or may be learned by practice of this disclosure.

It is to be understood that both the forgoing and the following descriptions are exemplary and explanatory only and are not intended to limit the claimed disclosure or application thereof in any manner whatsoever.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is an illustrative block diagram of a system based on a computer configured to execute one or more aspects of the functionality described herein.

FIG. 2 is an illustration of a computing machine configured to execute one or more aspects of the functionality described herein.

FIG. 3 represents the relationship between genomic intervals and informative variants.

FIG. 4 is a schematic representation of the steps of the method for determining absolute allele copy number and sample purity and sample ploidy from NGS sequencing data.

FIG. 5A is a genome-wide representation of normalized coverage profile for an exemplary sample. FIG. 5B (top panel) is a genome-wide representation of raw allele variant fraction (Naltᵢ/Dᵢ) measured in low-pass WGS data for an exemplary sample. FIG. 5B (bottom panel) is a genome-wide representation of the variant fraction estimated from raw data (top panel) for an exemplary sample. FIG. 5C (bottom panel) is a genome-wide representation of absolute copy number estimates obtained combining normalized coverage data (top left panel) with estimated variant fraction data (top right panel).

FIGS. 6A-6E provide an example of an analysis of a genomic dataset, plotted along numbered chromosomes, corresponding to a germline sample. The data was extracted from the 1000 Genome database, for one female individual. FIG. 6A shows the relative coverage, based on SNP array data; no copy number variants are visible. FIG. 6B shows the allele variant fractions across the genome, based on SNP array data. A deviation from the expected frequencies is observed at the end of chromosome 5 (indicated by a leftward arrow), indicative of germline mosaicism. In addition, three regions without heterozygotes are visible at the beginning of chromosome 8, on chromosome 18, and on the X chromosome (indicated with rightward arrows), representing LOH events. FIG. 6C shows the allele variant fractions across the genome, for the same sample, based on WGS data downsampled to 30× coverage. The events visible on FIG. 6B are still apparent, but less markedly. FIG. 6D shows the allele variant fractions across the genome, for the same sample, based on WGS data downsampled to 5X coverage. While the three LOH events are still apparent, the germline mosaicism on chromosome 5 is not visible. FIG. 6E shows the output of the model and methods described herein, corresponding to the embodiment with Hardy-Weinberg Equilibrium weights based on the population that best matched the sample, performed on the 5×WGS data. The model efficiently detects allele frequencies indicative of the germline mosaicism and the three LOH events.

FIG. 7 is a flowchart of a method for determining absolute allele copy number from NGS sequencing data.

FIG. 8 illustrates a next generation sequencing system according to certain embodiments of the present disclosure

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized, and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

The present disclosure is directed to, at least in part, a computer-implemented method to determine allele-specific copy number of a genomic interval, j, from genome sequencing a sample.

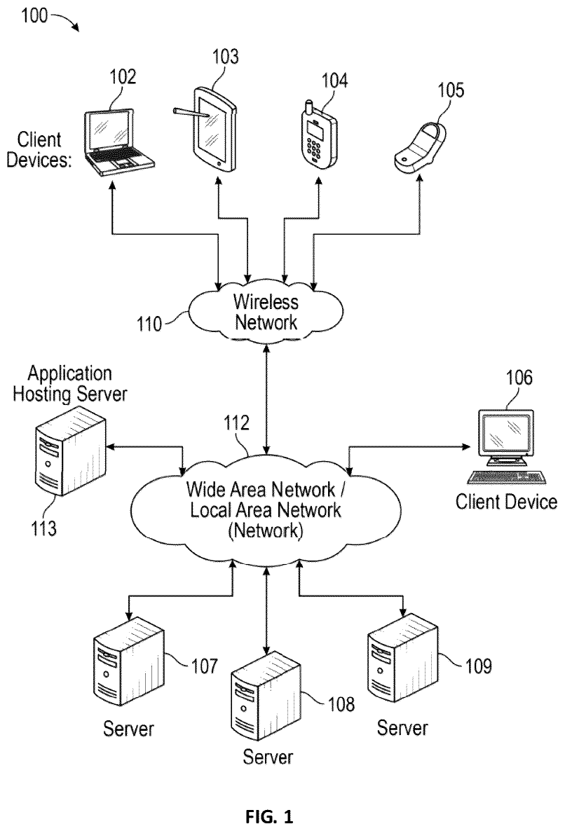

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

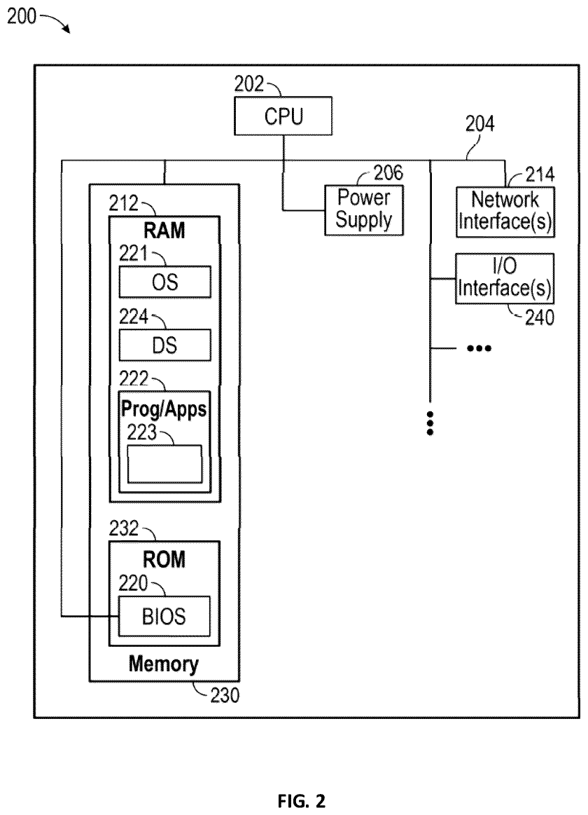

FIG. 2 illustrates a block diagram of an electronic device 200 that can implement one or more aspects of an apparatus, system and method for increasing mobile application user engagement (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can case processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as IOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games, receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. Thus, in various embodiments, a non-transitory computer readable medium may comprise instructions stored thereon that, when executed by a processing device, cause the processing device to carry out an operation (e.g., performing allele copy number estimation). In such an embodiment, the operation may be carried out on a singular device or between multiple devices (e.g., a server and a client device). A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G), 5th (5G) generation, Long Term Evolution (LTE) radio access for cellular systems, WLAN, Wireless Router (WR) mesh, and the like. Access technologies such as 2G, 2.5G, 3G, 4G, 5G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPV6. The Internet includes local area networks (LANs), Wide Arca Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

### Definitions

An “allele” refers to a variant form, typically relative to a reference genome, of a region of the genome. In diploid organisms, such as humans, each locus has typically two alleles, which can be identical (homozygous) or different (heterozygous). In the presence of copy number variation, the number of alleles can however differ from two.

A “copy number” refers to the number of copies in which a region is found in the genome of the sample.

An “allele specific copy number” or “allele copy number” refers to the number of copies of a specific allele within the genome of an organism.

“Mbp” is an abbreviation of “megabases,” which refers to a million base pairs in a genomc.

“Reference genome” refers to the DNA sequence that is used as the sequence of reference for the organism.

“Genomic locus” or in its plural form, “genomic loci,” refers to a region of at least 1 base pairs on a chromosome. The genomic locus location is defined by chromosome number and the genomic coordinates of the region start and end positions. A genomic locus may comprise a gene and/or genetic marker. In some cases, all genomic positions within a defined genome locus may have the same copy number, referred to as allele copy number.

“Minor-Allele Frequency” or “MAF” refers to a proportion of a less common allele at a genomic locus in a population.

An “alt sequence” or “alternative sequence” refers to a sequence that differs from the sequence present in the reference genome at the same genomic position.

“Coverage” or “sequencing depth” refers to the number of sequencing reads that have been aligned to a genomic position. Across a genomic region or the whole genome, “coverage” refers to the average number of reads that have been aligned to each position. In general, a genomic region with a higher coverage is associated with a higher reliability in downstream genomic characterization, in particular when calling variants. In target enrichment workflows, only a small subset of regions of interest in the whole genome is sequenced and it may therefore be reasonable to increase the sequencing depth without facing too significant data storage and data processing overhead. In some genomic analysis applications not requiring a high resolution along the genome, for instance, in detecting copy number alterations, low-pass (LP) coverage (1×-10×) or ultra-low-pass (ULP) coverage (<1×—not all positions are sequenced) may be more efficient in terms of information technology infrastructure costs, but these workflows require more sophisticated bioinformatics methods and techniques to support reliable results using the limited information afforded by low coverage data. Moreover, apart from the higher cost related to data storage and processing, the operational cost of an experimental NGS run, that is, loading a sequencer with samples for sequencing and covering the costs of a sequencing run, also needs to be optimized by balancing the coverage depth and the number of samples which may be assayed in parallel in routine clinical workflows. Indeed, a person having ordinary skill in the art will recognize next generation sequencers are still limited in the total number of reads that they can produce in a single experiment (i.e., in a given run). If the targeted coverage is lower, fewer reads per sample are required, and therefore a higher number of samples can be multiplexed within a next generation sequencing run. It is contemplated that as a result of the higher number of samples that can be multiplexed within the next generation sequencing run, lower coverage requirements result in reduced costs.

“Aligning” or “alignment” or “aligner” refers to mapping and aligning base-by-base, in a bioinformatics workflow, the sequencing reads to a reference genome sequence. For instance, in a targeted enrichment application where the sequencing reads are expected to map to a specific targeted genomic region in accordance with the hybrid capture probes used in the experimental amplification process, the alignment may be specifically searched relative to the corresponding sequence, defined by genomic coordinates such as the chromosome number, the start position and the end position in a reference genome. As known by those having ordinary skill in the art, in some embodiments “alignment” methods as employed herein may also comprise certain pre-processing steps to facilitate the mapping of the sequencing reads and/or to remove irrelevant data from the reads, for instance by removing non-paired reads, and/or by trimming the adapter sequence at the end of the reads, and/or other read pre-processing filtering means.

A “Markov model” is a statistical model used to model randomly changing systems. A “Hidden Markov Model” or “HMM” is a Markov model with unobserved (i.e., hidden) states. A hidden Markov model can be represented as the simplest dynamic Bayesian network. An HMM model enables to estimate a hidden state from an observation state, based on:1) pre-defined emission probabilities from the hidden states to the observation states; and/or 2) pre-defined transition probabilities between the hidden states themselves.

### Sample Characterization Method

The computer-implemented method to characterize a sample may comprise performing a series of steps, each step discussed in further detail herein. In some embodiments, the computer-implemented method may be operative to estimate allele-specific copy number for a genomic interval from genome sequencing from the sample. The characterization of a sample may include detection of allele-specific copy number and/or the sample ploidy and purity. Accordingly, “characterization of sample” is not intended to limit the scope of the workflow described herein.

In one embodiment, obtaining a whole-genome sequencing for the sample may comprise performing whole-genome sequencing. The whole-genome sequencing may be configured to obtain the whole-genome sequence at low coverage (low-pass whole-genome sequence). A person of ordinary skill in the art will recognize that any sequencing is contemplated, for example, any type of low-coverage genotyping data may be utilized. In some embodiments, the low-pass whole-genome sequencing may be configured to obtain the whole genome sequence at a coverage of about 1×, about 5×, and/or about 10×. However, any coverage of the whole genome may be utilized to carry out the systems and methods described herein.

The step of aligning the sequencing reads may be performed using any method known in the art. In one embodiment, the step of aligning the sequencing reads may comprise obtaining a read alignment file.

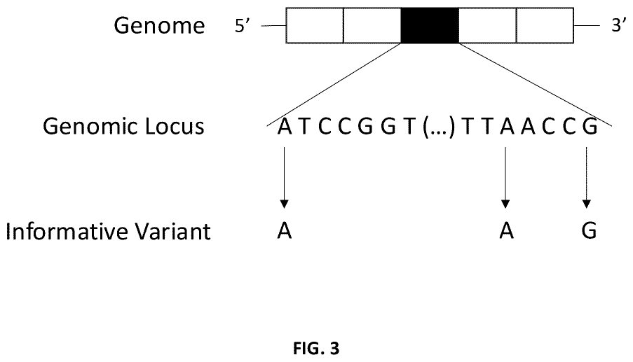

In one embodiment, the method may comprise selecting from the set of all genomic positions in the genome, variants that are likely heterozygous, hereafter referred to as informative variants, i. In an embodiment, identifying at least one informative variant may comprise filtering out variants that are homozygous or likely homozygous. A person of ordinary skill in the art will recognize homozygous variants refer to instances where the alleles from a same locus are identical, while heterozygous variants refer to instances where the alleles of the same locus differ. FIG. 3 represents the relationship between genomic intervals and informative variants.

In one embodiment, selecting variants that are informative variants i comprises querying a population variant database to identify variants that are frequently common in the population and thus likely polymorphic.

In one embodiment, selecting variants that are informative variants i comprises querying a population variant database to identify among multiple populations, the population that best matches the sample, and then identify variants that are polymorphic in the population. As a nonlimiting example, the multiple populations might correspond to populations of different geographic origins or ancestries in publicly available databases (e.g., Gnomad or the 1000 Genomes project) or in custom-built databases. In such embodiments, single nucleotide polymorphisms (SNPs) common to all populations in a collection of populations are first identified. The genotype of the sample at the selected positions may then be inferred from the whole-genome sequence data, and the population that best matches the sample based on the genotype at these positions is identified. It will be apparent to those skilled in the art that different methods can be used to assess the match between the sample and different populations.

In an embodiment, the method may comprise extracting the minor allele frequency (MAF) of a variant in a population, which may have been selected from multiple populations, to identify informative variants i. The MAF may be extracted from a publicly available population database, such as Gnomad or the 1000 Genomes projects, or a custom-built database, obtained by combining publicly available information, generating datasets, or a combination of both. As a nonlimiting example, the MAF could be extracted from observed frequencies among datasets analyzed by a given genomic platform. The method may further comprise filtering out any variants having a MAF value in a specified range, filtering out for example variants having a MAF below 30%. In various embodiments, the MAF threshold may be between 25% and 35%. In other embodiments, the MAF threshold may be between 10% and 35%. However, other MAF thresholds eliciting a similar effect are contemplated. Lowering the MAF threshold may increase the number of identified informative variants i, but may decrease the likelihood that they are heterozygous in the analyzed sample. Any threshold providing a high probability of the variant being heterozygous based on population genetics theory (e.g. Hardy-Weinberg equilibrium) is suitable. Persons of ordinary skill in the art will know that the MAF threshold can be slightly altered without significantly altering the outcome of the analyses.

In one embodiment, selecting variants that are informative variants i comprises obtaining and genotyping, using any method known to the person skilled in the art, a reference sample (e.g., a non-tumor sample in the case of a cancer) from the subject to determine and filter out any variant in the reference sample from the subject having a variant fraction (VAF) in a specified range. As a non-limiting example, variants having a VAF above a given threshold (e.g., 50%, 60%, or 75%) may be filtered out as being likely homozygous in the sample. In such embodiments, the VAF threshold may represent a level above which the homozygous status can be confidently established based on the level of noise expected from the data generation workflow and sequencing platform, using methods known to those skilled in the art.

For any of the informative variants, i, the method may comprise identifying in the read alignment file any number of reads that support an alt sequence (Naltᵢ) at the variant position. In one embodiment, the system may compute the number of reads in the alignment file that may support a finding of the alt sequence (Naltᵢ) for the informative variant i.

In some embodiments, the method may further comprise identifying a depth of sequencing (Dᵢ) according to the number of informative variants i, in the genome.

In one embodiment, estimating the variant fraction of i (VF_{U}[i]) may comprise estimating, with a Hidden Markov Model (HMM) along each chromosome, the most likely hidden VF_{U}[i] explaining the observed variant fraction of i (VFₒ[i]) given the observed NAltᵢ and Dᵢ.

In one embodiment, the observed states for the HMM along each chromosome comprise the raw variant fractions NAltᵢ/Dᵢ for the list of informative loci.

In one embodiment, hidden states for the HMM along each chromosome comprise a number of discretized variant fractions. Various resolutions for the discretized VF_{U} values may be used by the Hidden Markov Model. In general, the lower the number of states, the faster the computation but the less accurate the filter. In some embodiments, the number of hidden states of the HMM filter is at least nine VF_{U} value estimates (VF_{U}=10%, 20%, . . . , 90%), or around 19 VF_{U} value estimates (VF_{U}=5%, 10%, 15% . . . , 95%), or around 99 VF_{U} value estimates (VF_{U}=1%, 2%, . . . , 99%). Other embodiments are also possible. It will be apparent to those skilled in the art that the optimal number of hidden states is a function of the available sequencing coverage (as finer granularity may be enabled at higher coverage) and the available computing resources (as the complexity increases in a quadratic relationship to the number if hidden states). The number of hidden states may also be altered depending on the desired application. As nonlimiting examples, tests for LOH in a germline context may achieve high sensitivity with nine hidden states. However, as a nonlimiting example, precise estimates of allele-specific copy numbers in a somatic context where fractions are low may require a higher number of hidden states. Thus, in instances where the instant method is utilized for tumor content analysis, granularity may improve the results. In such an instance, the result may be optimized by increasing the number of hidden states, for example to ninety-nine states at 1% discretization. However, the number of hidden states may be modified according to the use case, desired granularity, and available computing power.

The HMM model may employ a transition probability (p_{switch}) representing the probability that two subsequent variants, i−1 and i, along a chromosome, with variant fractions VFₒ[i−1] and VFₒ[i], respectively, are not associated to the same hidden state (i.e., VF_{U}[i−1] for VFₒ[i−1] and VF_{U}[i] for VFₒ[i], where VF_{U}[i−1] and VF_{U}[i] are two different hidden states).

In one embodiment, emission probabilities of the HMM are defined as:

$$P ( {NAlt}_{i} , {D}_{i} \vert {VF}_{U} [ i ] ) = {P}_{hom , alt} ( i ) + {P}_{h o m , r e f} ( i ) + {P}_{het} ( i )$$

and where:
P_{hom,alt} is the weighted probability of obtaining NAltᵢ given i being homozygous alt;
P_{hom,ref} is the weighted probability of obtaining NAltᵢ given i being homozygous reference; and
Pₕₑₜ is the weighted probability of obtaining NAltᵢ given i being heterozygous.

In one embodiment, determining the probability of observing the variant fraction may further comprise performing:

$${P}_{hom , alt} ( i ) = {W}_{hom , alt} \cdot Binomial ( {NAlt}_{i} , {D}_{i} \vert p = 1 )$$

$${P}_{h o m , r e f} ( i ) = {W}_{hom , ref} \cdot Binomial ( {NAlt}_{i} , {D}_{i} \vert p = 0 )$$

$${P}_{het} ( i ) = 1 / 2 \cdot {W}_{het} [ Binomial ( {NAlt}_{i} , {D}_{i} \vert p = {VF}_{U} [ i ] + Binomial ( {NAlt}_{i} , {D}_{i} \vert p = 1 - {VF}_{U} [ i ] )$$

and where:
W_{hom,alt}, W_{hom,ref} and Wₕₑₜ are the probability weighting factors. The sum of all probability weighting factors (W_{hom,alt}+W_{hom,ref}+Wₕₑₜ) is equal to 1.

In one embodiment, the Binomial distribution may be replaced by other distributions such as Beta Binomial. In one embodiment, the weights can be defined empirically, for example, based on the most likely genotype for the locus inferred from prior information obtained for the individual from whom the sample was obtained. In another embodiment, weights can also be defined based on modeling of available data for the selected population and the locus, with or without selecting the population that best matches the subject. For example, weights can be inferred from the MAF observed for variant i using a variant population database. In one embodiment, Hardy-Weinberg Equilibrium assumptions may be used to determine the weights given the observed MAF at i, such that:

$${W}_{hom , alt} = {MAF}_{i} * {MAF}_{i}$$

$${W}_{hom , ref} = ( 1 - {MAF}_{i} ) * ( 1 - {MAF}_{i} )$$

$${W}_{het} = 2 * ( 1 - {MAF}_{i} ) * {MAF}_{i} .$$

In one embodiment, the Hardy-Weinberg Equilibrium with inbreeding may be used. The inbreeding factor F may be optimized based on the sample. In a nonlimiting example, the F value that optimizes the fit of the sample data might be selected among F values varied from 0 to 0.2 in 0.01 increments. In this example, the upper bound of 0.2 is chosen to exceed inbreeding coefficients observed in most human populations. The weights given the observed MAF at I are as follow for a model with inbreeding:

$${W}_{hom , alt} = {MAF}_{i} * {MAF}_{i} + F * ( 1 - {MAF}_{i} ) * {MAF}_{i}$$

$${W}_{hom , ref} = ( 1 - {MAF}_{i} ) * ( 1 - {MAF}_{i} ) * ( 1 - F )$$

$${W}_{het} = 2 * ( 1 - {MAF}_{i} ) * {MAF}_{i} + F * ( 1 - {MAF}_{i} ) * {MAF}_{i}$$

However, in still another embodiment, the weights may be calculated using other assumption models that a person of ordinary skill in the art may contemplate. Of course, other modeling methods may be utilized.

Given a transition and emission probability, a set of VFₒ[i] (NAlti/Di), and an initial probability, the HMM may then be adapted to employ an algorithm configured to infer the posterior probability distribution of the hidden states VF_{U}[i] at each informative variant i given the observation VFₒ[i]. The algorithm may be a forward-backward algorithm (Collins, Encyclopedia of Biometrics 2009) adapted to compute “forwards” and “backwards” probabilities in the Hidden Markov Model and to infer the posterior probability distribution of the hidden states VF_{U}[i] at each informative variant i given the observation VFₒ[i]. In an embodiment, the transition and emission probability, a set of VFₒ[i], and an initial probability may be used by a forward-backward algorithm or Viterbi algorithm (or similar suitable model) to infer the most likely variant hidden state VF_{U}[i] at each informative locus i.

In one embodiment, the method may be configured to output a list of genomic loci, wherein genomic loci are defined as regions along each chromosome for which all informative variants i within the genomic loci have the same VF_{U}[i].

In an embodiment, the initial probability is chosen that the hidden states have equally probabilities at the first position (i=1), is 0.5. Any other initial probability may be chosen, and it will be apparent to those skilled in the art that the initial probability does not affect the outcome of the model.

In some embodiments, the whole genome may be divided into at least two intervals. In one embodiment, each of the at least two intervals may be a non-overlapping, contiguous genomic interval. In some embodiments, the interval size may be within a predetermined range. In other embodiments, the interval size may be determined by a predefined value. As a non-limiting example, the interval size may be between one and three Mbp, inclusive. It is contemplated that the interval size may be determined according to a desired resolution of genome analysis. As a nonlimiting example, the predetermined size/range may be a function of one or more aspects, including, but not limited to, (i) the resolution desired by the user for CNV calling at the final stage (e.g., fewer large intervals will give lower resolution, while more smaller intervals will give higher resolution), and (ii) the actual bounds of the available coverage (e.g., the higher the coverage, the more informative are individual SNPs and the smaller the intervals can reasonably be).

A person of ordinary skill in the art will recognize that genomes and chromosomal arms comprise a predefined number of intervals and an interval size may be defined as a function of the coverage. As such, the number of intervals may be defined according to methods known in the art.

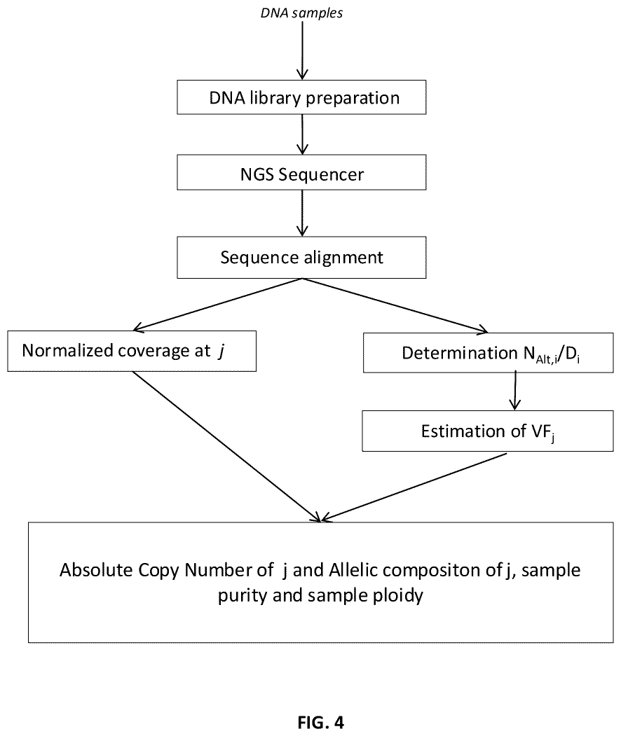

The method may further comprise estimating for each genomic interval j in a sample a sample-level variant fraction (VF_{U}[j]) of the genomic interval j by modeling the information obtained from low-pass whole-genome sequencing data for the normalized coverage of at least one informative variant, i, included in the genomic interval j. FIG. 4 is a schematic representation of the steps of a method for determining absolute allele copy number and sample purity and sample ploidy from NGS sequencing data.

Estimating the sample variant fraction VF_{U}[j] may comprise performing an iterative method comprising the steps of:
a. Assigning a value, x, between 0 and 1 to the variant fraction VF_{U}[j];
b. For each informative variants i in the genomic interval j, determining the probability of observing a variant fraction VF_{U}[j] given the number of reads supporting the alt sequence (NAltᵢ) and the depth of the sequencing at i (Dᵢ), wherein:

$$P ( NA l {t}_{i} , {D}_{i} \vert {VF}_{U} [ j ] ) = {P}_{hom , alt} ( i ) + {P}_{h o m , r e f} ( i ) + {P}_{het} ( i )$$

and where:
P_{hom,alt} is the weighted probability of obtaining NAltᵢ given i being homozygous alt;
P_{hom,ref} is the weighted probability of obtaining NAltᵢ given i being homozygous reference;
Pₕₑₜ is the weighted probability of obtaining NAltᵢ given i being heterozygous.

In one embodiment, determining the probability of observing the variant fraction may further comprise performing:

$${P}_{hom , alt} ( i ) = {W}_{hom , alt} \cdot Binomial ( {NAlt}_{i} , {D}_{i} \vert 1 )$$

$${P}_{h o m , r e f} ( i ) = {W}_{hom , ref} \cdot Binomial ( {NAlt}_{i} , {D}_{i} \vert 0 )$$

$${P}_{het} ( i ) = 1 / 2 \cdot {W}_{het} [ Binomial ( {NAlt}_{i} , {D}_{i} \vert {VF}_{U} [ j ] ) = x ) + Binomial ( {NAlt}_{i} , {D}_{i} \vert {VF}_{U} [ j ] ) = 1 - x )$$

and where:
W_{hom,alt}, W_{hom,ref} and Wₕₑₜ are the probability weighting factors. The sum of all probability weighting factors (W_{hom,alt}+Whom,ref+Wₕₑₜ) is equal to 1.

In one embodiment, the Binomial distribution may be replaced by other distributions such as a Beta Binomial.

In one embodiment, the weights can be defined empirically, for example, based on the most likely genotype for the locus, inferred from prior information obtained for the individual from whom the sample was obtained. In another embodiment, weights can also be defined based on modeling of available data for the population selected for the sample and the locus. For example, weights can be inferred from the MAF observed for variant i using the selected variant population database. In one embodiment Hardy-Weinberg Equilibrium assumptions may be used to determine the weights given the observed MAF at i. and where:

$${W}_{hom , alt} = {MAF}_{i} * {MAF}_{i}$$

$${W}_{hom , ref} = ( 1 - {MAF}_{i} ) * ( 1 - {MAF}_{i} )$$

$${W}_{het} = 2 * ( 1 - {MAF}_{i} ) * {MAF}_{i}$$

However, in still another embodiment, the weights may be calculated using other assumption models that a person of ordinary skill in the art may desire. Of course, other modeling methods may be utilized. For example, uniform, normal, t-test, Bernoulli, Poisson, or other distributions that a person of ordinary skill may desire.
c. For each genomic interval j, determine the likelihood (h) of VF_{U}[j] being x wherein:

$$h = {\prod }_{i = i}^{N} P ( NAlti , Di \vert {VF}_{U} [ j ] ) = x )$$

d. Repeat steps b to c using a value of x between 0 and 1 different from the one used in a. Compare the likelihood (h) obtained for the two values of x and retain the value of x that yields the highest likelihood.
e. Repeat d until value x of VF_{U}[j] that maximizes the likelihood (h) is found using methods known to the person skilled in the art.

In one embodiment, normalized coverage may be computed for all intervals j in the sample using the read alignment file and methods known to the person skilled in the art. For example, in an embodiment, normalized coverage may comprise normalizing per sample, by dividing the coverage data signal by the mean coverage signal for the whole sample, in order to account for differences introduced by data generation and sequencing of the sample. Indeed, variation in the origin of the source of the DNA, for example, whether it was isolated from a fresh frozen sample, a liquid sample, or a formalin-fixed paraffin-embedded (FFPE) sample; the details of the library preparation, for example, the temperatures, buffers, PCR cycles, and the like; and the sequencing platform, may all introduce coverage noises (e.g. Aird, D., Ross, M. G., Chen, W. S., Danielsson, M., Fennell, T., Russ, C., Jaffe, D. B., Nusbaum, C. and Gnirke, A., 2011. Analyzing and minimizing PCR amplification bias in Illumina sequencing libraries. Genome biology, 12(2), pp. 1-14; Heinrich, V., Stange, J., Dickhaus, T., Imkeller, P., Krüger, U., Bauer, S., Mundlos, S., Robinson, P. N., Hecht, J. and Krawitz, P. M., 2012. The allele distribution in next-generation sequencing data sets is accurately described as the result of a stochastic branching process. Nucleic acids research, 40(6), pp. 2426-2431). In the presence of such noises, the normalization may improve detection of the true signal. Normalized coverage may also comprise normalization by GC content to apply a GC-bias correction. Other embodiments are also possible, such as bin-wise normalization, linear and nonlinear least squares regression, GC LOESS (GC normalization), LOWESS, PERUN (normalization per sample), RM, GCRM, cQn and/or combinations thereof.

In some embodiments, the method may further comprise modeling, according to any of normalized coverage and the estimated VF_{U}[i] for any of the genomic loci i, the allele specific copy number (defined by allele composition and the total number of copies of each allele) for all genomic loci i given an estimated sample purity and sample ploidy, using methods known to the person skilled in the art.

In one embodiment, the output may be selected from a group consisting of the absolute copy number for at least one genomic locus i, an allelic composition for at least one genomic locus i (i.e., list and copy number for all alleles at the locus), the sample purity, the sample ploidy, and combinations thereof. The absolute copy number may, in some embodiments, be transformed into the allelic composition. It will be evident to those skilled in the art that the method can therefore be used to estimate, for example, the tumor purity and tumor ploidy of a cancer sample from low-pass whole-genome sequencing data.

In one embodiment, the method may further comprise identifying from a set of possible genomic events what is the most likely to explain the observed allele-specific copy number for at least one genomic locus i.

In some embodiments, the set of possible genomic events comprises any of duplication, deletion, loss of heterozygosity, and combinations thereof. Of course, the set of possible genomic events may comprise any genomic event that may be contemplated for a given analysis.

In one embodiment, determining of the genomic events can be done by parsimony. For example, a scenario of genomic events might be selected from among a set of genomic events explaining equally well the observed data as the scenario involving the minimum number of genomic events.

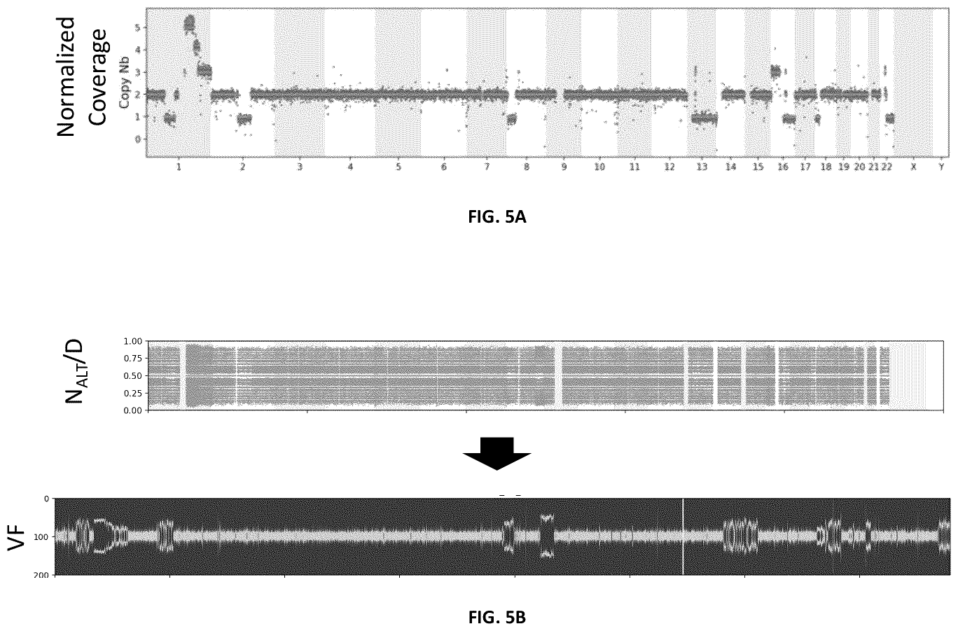

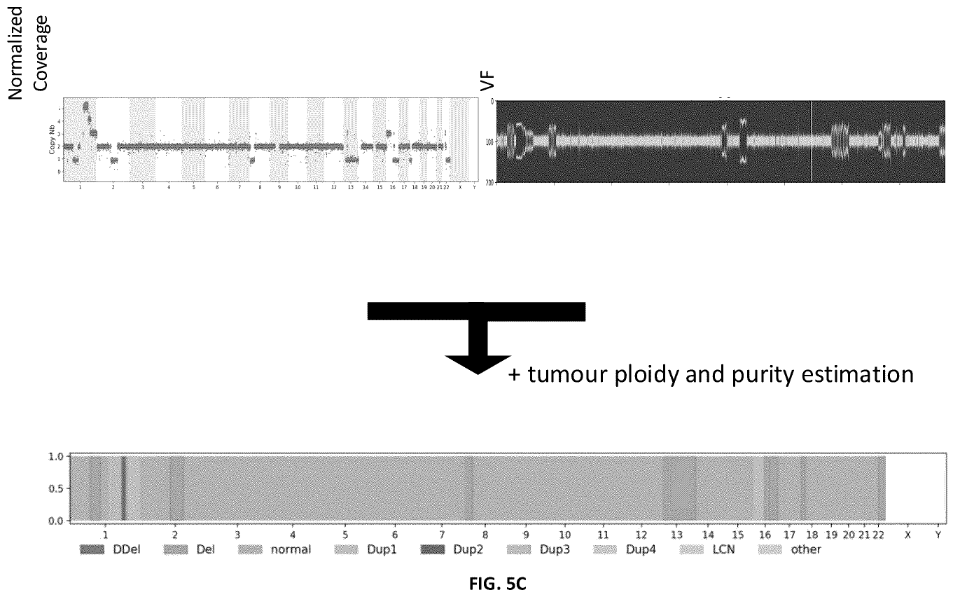

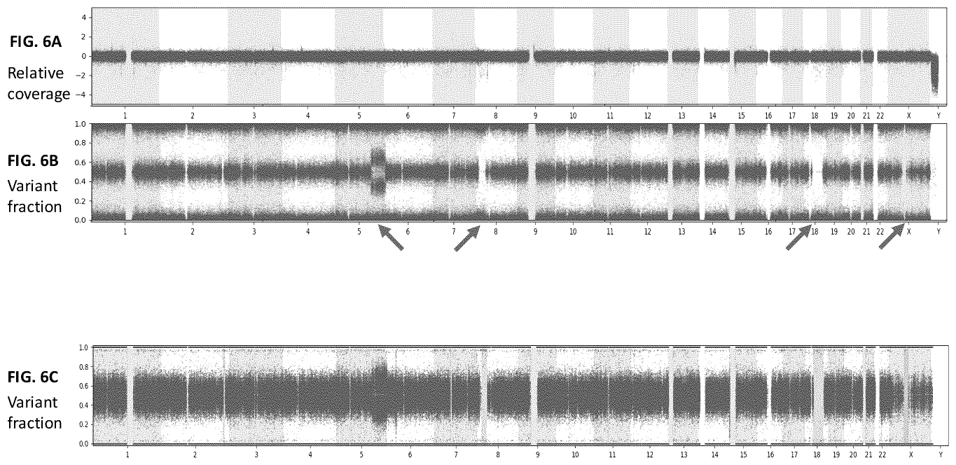

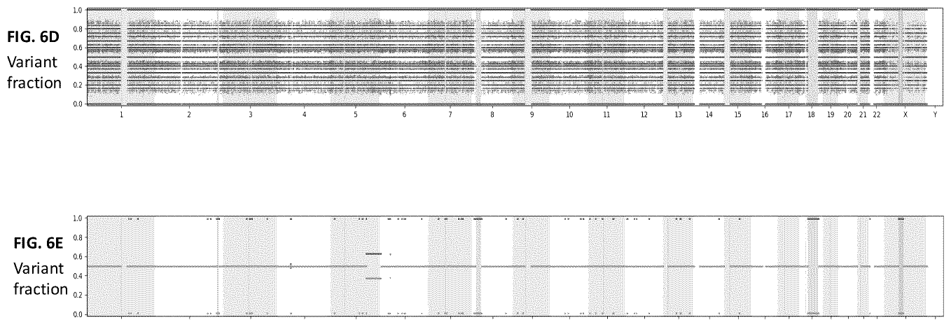

Referring to FIGS. 5A-5C and FIGS. 6A-6E, the figures and data provided therein are for illustrative purposes only and are not intended to limit the scope of the present disclosure. Variations of the depicted figures and data may be contemplated without departing from the spirit and scope of the disclosure.

FIG. 5A is a genome-wide representation of normalized coverage profile for an exemplary sample. FIG. 5B (top panel) is a genome-wide representation of raw allele variant fraction (Naltᵢ/Dᵢ) measured in low-pass WGS data for an exemplary sample. FIG. 5B (bottom panel) is a genome-wide representation of the variant fraction estimated from raw data (top panel) for an exemplary sample. FIG. 5C (bottom panel) is a genome-wide representation of absolute copy number estimates obtained combining normalized coverage data (top left panel) with estimated variant fraction data (top right panel).

FIGS. 6A-6E provide an example of an analysis of a genomic dataset, plotted along numbered chromosomes, corresponding to a germline sample. The data was extracted from the 1000 Genome database, for one female individual. FIG. 6A shows the relative coverage, based on SNP array data; no copy number variants are visible. FIG. 6B shows the allele variant fractions across the genome, based on SNP array data. A deviation from the expected frequencies is observed at the end of chromosome 5 (indicated by a leftward arrow), indicative of germline mosaicism. In addition, three regions without heterozygotes are visible at the beginning of chromosome 8, on chromosome 18, and on the X chromosome (indicated with rightward arrows), representing LOH events. FIG. 6C shows the allele variant fractions across the genome, for the same sample, based on WGS data downsampled to 30X coverage. The events visible on FIG. 6B are still apparent, but less markedly. FIG. 6D shows the allele variant fractions across the genome, for the same sample, based on WGS data downsampled to 5× coverage. While the three LOH events are still apparent, the germline mosaicism on chromosome 5 is not visible. FIG. 6E shows the output of the model and methods described herein, corresponding to the embodiment with Hardy-Weinberg Equilibrium weights based on the population that best matched the sample, performed on the 5×WGS data. The model efficiently detects allele frequencies indicative of the germline mosaicism and the three LOH events.

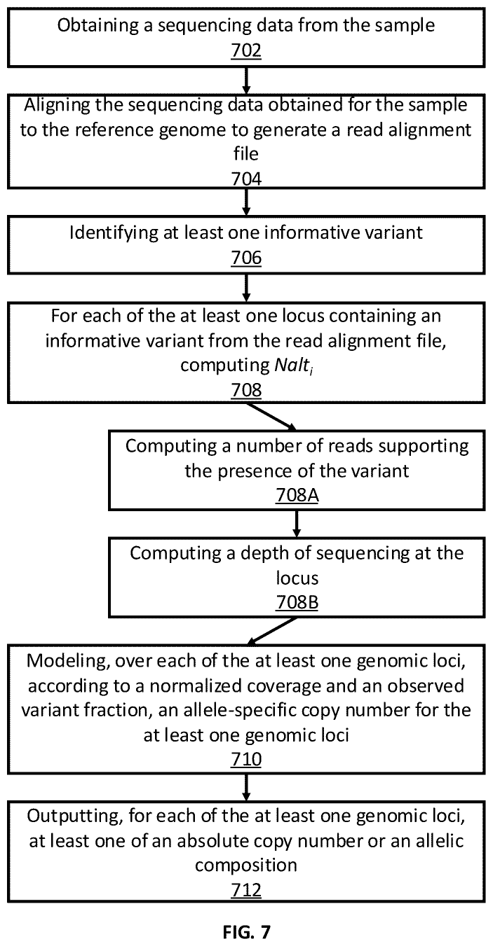

As disclosed above, and as shown in FIG. 7, the present disclosure provides a computer-implemented method for characterization of a sample from low-coverage genotyping data. Although the method described herein may be utilized with low-coverage genotyping data, the method may be utilized with other suitable genotyping data. The method may comprise a step 702 of obtaining a sequencing data from the sample. The sample may be a DNA sample, for example, DNA from a tumor, tumor cell-free DNA (cfDNA), a fresh-frozen tissue (FFT) or a formalin-fixed paraffin-embedded (FFPE) sample. The sample may represent a germline sample and may comprise DNA isolated from blood, saliva, or another tissue. As a nonlimiting example, the sample comprises cfDNA isolated from a liquid biopsy. The method may further comprise a step 704 of aligning the sequencing data obtained for the sample to the reference genome to generate a read alignment file. In an embodiment, the method comprises a step 706 of identifying at least one informative variant. The step of identifying the at least one informative variant may include filtering out those that are likely homozygous. In another embodiment, the step of identifying the at least one informative variant further comprises identifying variants that are polymorphic in a population database. The method may comprise a step 708 of, for each of the at least one locus containing an informative variant from the read alignment file, computing NAltᵢ, comprising substep 708A of computing a number of reads supporting the presence of the variant, and substep 708B of computing a depth of sequencing at the locus. In an embodiment, the method comprises a step 710 of modeling, over each of the at least one genomic loci, according to a normalized coverage and an observed variant fraction, an allele-specific copy number for the at least one genomic loci; and/or a step 712 of outputting, for each of the at least one genomic loci, at least one of an absolute copy number or an allelic composition. In one embodiment, step 710 further comprises estimating with a Hidden Markov Model (HMM) a most likely hidden variant fraction for the at least one genomic locus, wherein the observed states comprise the number of reads supporting the variant and the sequencing depth of the locus.

### Genomic Analysis System

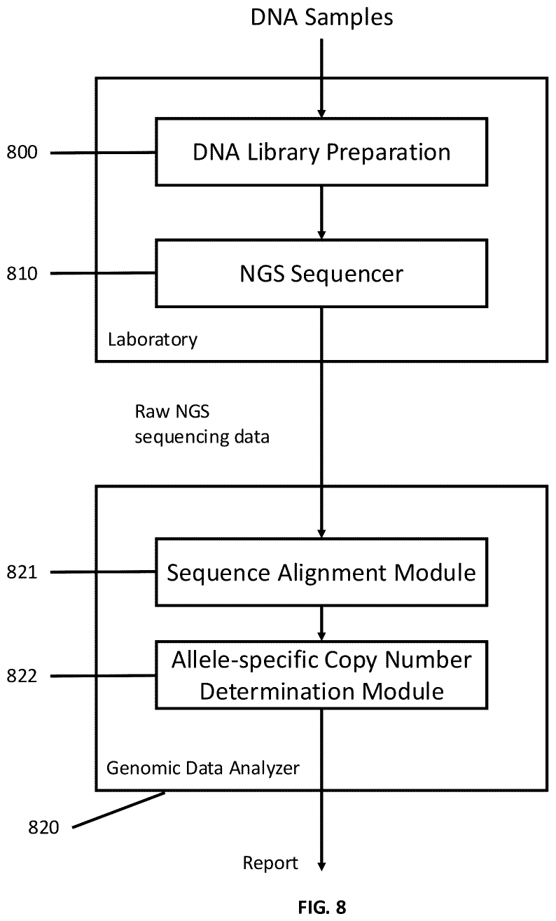

The proposed methods and systems will now be described by an exemplary genomic analysis system and workflow described with further detail to reference to FIG. 8. As will be apparent to those skilled in the art of DNA analysis, a genomic analysis workflow comprises preliminary experimental steps to be conducted in a laboratory (also known as the “wet lab”) to produce DNA analysis data, such as raw sequencing reads in a next-generation sequencing workflow, as well as subsequent data processing steps to be conducted on the DNA analysis data to further identify information of interest to the end users, such as the detailed identification of DNA variants and related annotations, with a bioinformatics system (also known as the “dry lab”). Depending on the actual application, laboratory setup and bioinformatics platforms, various embodiments of a DNA analysis workflow are possible. FIG. 8 describes an example of an NGS system comprising a wet lab system wherein DNA samples are first experimentally prepared with a DNA library preparation protocol 800 which may produce, adapt for sequencing and amplify DNA fragments to facilitate the processing by an NGS sequencer 810. In a next generation sequencing workflow, the resulting DNA analysis data may be produced as a data file of raw sequencing reads in the FASTQ format. The workflow may then further comprise a dry lab genomic data analyzer 820 which takes as input the raw sequencing reads for a pool of DNA samples prepared according to the proposed methods and applies a series of data processing steps to characterize certain genomic features of the input samples.

As illustrated in FIG. 8, the genomic data analyzer 820 may comprise a sequence alignment module 821, which compares the raw NGS sequencing data to a reference genome, for instance the human genome in medical applications, or an animal genome in veterinary applications. In a conventional genomic data analyzer system, the resulting alignment data may be further filtered and analyzed by a variant calling module (not represented) to retrieve variant information such as SNP and INDEL polymorphisms. The variant calling module may be configured to execute different variant calling algorithms. The resulting detected variant information may then be output by the genomic data analyzer module 820 as a genomic variant report for further processing by the end user, for instance with a visualization tool, and/or by a further variant annotation processing module (not represented). In a possible embodiment, the genomic data analyzer system 820 may comprise automated data processing modules such as an allele-specific copy number determination module 822 to determine, for example, for each of at least one genomic loci, at least one of an absolute copy number or an allelic composition, which may then be reported to the end user, for instance with a visualization tool, or to another downstream process (not represented). The proposed genomic data analyzer 820 may be adapted in the SOPHIA Genetics Data Driven Medicine (DDM) genomic analysis software platform to implement the proposed method as a method for improved allele-specific copy number estimation over the prior art NGS workflows.

### Data Processing Workflow

The genomic data analyzer 820 may process the sequencing data to produce a genomic data analysis report by employing and combining different data processing methods.

The sequence alignment module 821 may be configured to execute different alignment algorithms. Standard raw data alignment algorithms such as Bowtie2 or BWA that have been optimized for fast processing of numerous genomic data sequencing reads may be used, but other embodiments are also possible. The alignment results may be represented as one or several files in BAM or SAM format, as known to those skilled in the bioinformatics art, but other formats may also be used, for instance compressed formats or formats optimized for order-preserving encryption, depending on the genomic data analyzer 820 requirements for storage optimization and/or genomic data privacy enforcement.

The genomic data analyzer 820 may be a computer system or part of a computer system including a central processing unit (CPU, “processor” or “computer processor” herein), memory such as RAM and storage units such as a hard disk, and communication interfaces to communicate with other computer systems through a communication network, for instance the internet or a local network. Examples of genomic data analyzer computing systems, environments, and/or configurations include, but are not limited to, personal computer systems, server computer systems, handheld or laptop devices, multiprocessor systems, microprocessor-based systems, set top boxes, programmable consumer electronics, network PCs, minicomputer systems, mainframe computer systems, graphical processing units (GPU), and the like. In some embodiments, the computer system may comprise one or more computer servers, which are operational with numerous other general purpose or special purpose computing systems and may enable distributed computing, such as cloud computing, for instance in a genomic data farm. In some embodiments, the genomic data analyzer 820 may be integrated into a massively parallel system. In some embodiments, the genomic data analyzer 820 may be directly integrated into a next generation sequencing system.

The genomic data analyzer 820 computer system may be adapted in the general context of computer system-executable instructions, such as program modules, being executed by a computer system. Generally, program modules may include routines, programs, objects, components, logic, data structures, and so on that perform particular tasks or implement particular abstract data types. As is well known to those skilled in the art of computer programming, program modules may use native operating system and/or file system functions, standalone applications; browser or application plugins, applets, etc.; commercial or open source libraries and/or library tools as may be programmed in Python, Biopython, C/C++, or other programming languages; and/or custom scripts, such as Perl or Bioperl scripts.

Instructions may be executed in distributed cloud computing environments where tasks are performed by remote processing devices that are linked through a communications network. In a distributed cloud-computing environment, program modules may be located in both local and remote computer system storage media including memory storage devices.

It is thus understood that methods described herein are computer-implemented methods. The NGS sequencer 810, the sequence alignment module 821, the allele-specific copy number determination module 822, and/or the genomic data analyzer 820, generally, may embody the electronic device 200 and/or components thereof. Accordingly, in one embodiment each of the modules depicted in FIG. 8 may be configured as standalone computer-executable instructions within the electronic device 200 and/or any one of the client devices 102-106 or servers 107-113. In an embodiment, one or more of the NGS sequencer 810, the sequence alignment module 321, the allele-specific copy number determination module 822, and/or the genomic data analyzer 820 may be configured as standalone hardware components. Improvements discussed herein to the allele-specific copy number determination workflow may facilitate improvements in both the adaptability of the method, but also the performance and efficacy of the hardware components executing the various steps of said method.

Embodiments of the present invention include apparatuses, systems, and methods implementing the Engine (as described in reference to FIGS. 1 and 2). Embodiments of the present invention may be implemented on one or more of client devices 102-106, which are communicatively coupled to servers including servers 107-109. For example, the client devices 102-106 may represent desktop computers (or other computers available in a clinical setting) utilized by clinicians in analyzing genomic data (e.g., in allele-specific copy number determination), while servers 107-109 may represent servers managed by the genomic data analysis platform. In such an embodiment, the majority of analysis or processing involved with a given genomic analysis workflow (e.g., that of FIG. 7) may be executed on the servers 107-109, wherein the request for such analysis or processing may originate from the client devices 102-105 (e.g., a clinician making a genomic data analysis request via a dry lab computer). In particular, software aspects of the Engine may be implemented in the program 223. In an embodiment, the methods disclosed herein, for example, as related to the determination of allele-specific copy numbers, may be embodied in a program 223 in the form of computer-executable instructions. The program 223 may be implemented on one or more client devices 102-106, one or more servers 107-109, and 113, or a combination of one or more client devices 102-106, and one or more servers 107-109 and 113. As a nonlimiting example, steps 702-712 may be executed on the one or more servers 107-109 and 113, wherein the request to begin such steps may originate from the one or more client devices 102-106.

In an embodiment, the method described herein may be integrated in a drylab workflow, where the data is generated by the users using a sequencing method of their choice, or a bundled solution, where the data is produced, by the user, using a provided kit. In either case, the user may then further upload their data into a platform, for example, the SOPHIA Genetics DDM.

In various embodiments, the method described herein may be utilized by a platform (e.g., the SOPHIA Genetics DDM or other software platforms designed for managing and analyzing genomic data in clinical settings), wherein said platform comprises various computer-executable instructions and/or modules configured to carry out specific analytical tasks (e.g., detecting variants, annotating oncogenic or pathogenic variants, classifying cancer mutations, identifying pathogenic variants, such as SNPs and Indels, that could, for example, be potential causes of HRD, and other specific tasks of the like). Accordingly, each analytical task that utilizes absolute copy number or allelic composition information would trigger the computer-executable instructions comprising the method described herein and/or the allele-specific copy number determination module 822. For example, selection of a given task on the frontend of the platform may then trigger pipelines that, when absolute copy number an allelic composition information is useful, would include the computer-implemented method described herein.

The solution as disclosed herein aims to provide suitable means for clinical determination of allele copy numbers. However, the versatility of the method and its steps extend beyond this scope, allowing for adaptation to various alternative use cases. While clinical determination of allele copy numbers is an exemplary embodiment, these methods demonstrate applicability across a spectrum of potential use cases, highlighting their flexibility and broad utility.

Accordingly, the method described herein is configured to improve upon conventional relativistic copy number analysis, which is insufficient for determination of absolute copy number changes of CNVs and other allele copy number changes, including LOH, which cannot accurately be determined from standard coverage depth analysis of IpWGS data using conventional methods. The proposed method may enable determination of allele copy numbers from lpWGS alone and without prior knowledge of common karyotypes to predict allele copy number and infer purity and ploidy are currently missing.

Further, since low pass sequencing generally generates fewer reads, the steps of sequencing themselves take less time. Accordingly, this reduces the time spent on related tasks, like data processing, alignment, storage, and the like. Yet further, the use of the method described herein permits the resource savings associated with lpWGS (e.g., decreased use of chemical reagents and consumables for DNA extraction from the samples) while providing accurate determination of allele copy numbers.

The workflow for the solution and illustrative depictions thereof are presented in at least FIGS. 3-8.

These and other aspects, features, and advantages of the present disclosure will become more readily apparent from the following drawings and the detailed description of the embodiments above.

The workflow described herein may be executed and/or used in connection with any suitable machine learning, artificial intelligence, and/or neural network methods. For example, the machine learning models may be one or more classifier and/or neural network. However, any types of models may be utilized, including regression models, reinforcement learning models, vector machines, clustering models, decision trees, random forest models, Bayesian models, and/or Gaussian mixture models. In addition to machine learning models, any suitable statistical models and/or rule-based models may be used.

The workflow disclosed herein may be incorporated in bioinformatic solutions to analyze whole-genome sequence data, whole-exome sequence data, or large-panel sequence data. In a nonlimiting example, the workflow may be incorporated in a bioinformatic workflow that takes as input sequence reads, for example, in FASTQ format, process the reads to align them to a reference genome, and use algorithms to call variants from the aligned reads. Such a bioinformatic workflow may be provided to users as standalone tools, which may be installed and executed on a local computer or may be executed on a centralized or a cloud-computing server, for example, after receiving a request from a user interface, such as a web application. In other embodiments, the bioinformatic workflow may be executed on a cloud-computing server, and the analysis requested might be received from a user-interactive genomic platform, such as SOPHIA DDM™.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the claims.

What is claimed is:

1. A computer-implemented method for characterization of a sample from low-coverage genotyping data, the method comprising:
obtaining a sequencing data from the sample;
aligning the sequencing data obtained from the sample to a reference genome to generate a read alignment file;
identifying at least one informative variant;
for each of at least one locus containing an informative variant from the read alignment file, computing NAltᵢ, comprising:
computing a number of reads supporting a presence of the variant, and
computing a depth of sequencing at the locus;
modeling, over each of at least one genomic loci, according to a normalized coverage and an observed variant fraction, an allele-specific copy number for the at least one genomic loci; and
outputting, for each of the at least one genomic loci, at least one of an absolute copy number or an allelic composition.

2. The computer-implemented method of claim 1, wherein identifying the at least one informative variant involves filtering out those that are likely homozygous.

3. The computer-implemented method of claim 2, wherein identifying the at least one informative variant further comprises identifying variants that are polymorphic in a population database.

4. The computer-implemented method of claim 3, wherein the population database is selected among multiple possible population databases as the one that most closely matches the sample genotype.

5. The computer implemented method of claim 3, wherein identifying the at least one informative variant comprises filtering out variants having a minor allele frequency (MAF) below 30% in a reference population.

6. The computer implemented method of claim 3, wherein identifying the at least one informative variant comprises filtering out variants having a minor allele frequency (MAF) below a threshold in a range of 5% to 40% in a reference population.

7. The computer-implemented method of claim 1, wherein modeling the allele-specific copy number comprises estimating with a Hidden Markov Model (HMM) a most likely hidden variant fraction for the at least one genomic locus, wherein the observed states comprise the number of reads supporting the variant and the sequencing depth of the locus.

8. The computer-implemented method of claim 7, wherein the hidden states are given different weights based on a binomial distribution, a Beta binomial distribution, a uniform distribution, a normal distribution, a Poisson distribution, or another pre-existing distribution.

9. The computer-implemented method of claim 7, wherein the hidden states are given different weights based on the Hardy-Weinberg Equilibrium expected based on a variant frequency in a reference population.

10. The computer-implemented method of claim 9, wherein the weights based on Hardy-Weinberg Equilibrium are corrected to account for inbreeding in the population.

11. The computer-implemented method of claim 10, wherein an inbreeding coefficient is optimized based on the sample.

12. The computer-implemented method of claim 1, further comprising determining at least one genomic event corresponding to the absolute copy number and the allelic composition.

13. The computer-implemented method of claim 1, further comprising identifying loss-of-heterozygosity events.

14. The computer implemented method of claim 1, wherein the sequencing data is derived from whole-genome sequencing, and wherein the whole-genome sequencing is low-pass whole-genome sequencing.

15. The computer implemented method of claim 1, wherein the sequencing data is derived from whole-exome sequencing or large panel sequencing, and wherein the sequencing coverage is below 10×.

16. The computer-implemented method of claim 1, wherein the sample represents a germline sample and comprises DNA isolated from blood, saliva, or another tissue.

17. The computer-implemented method of claim 1, wherein the sample comprises cell-free DNA (cfDNA) isolated from a liquid biopsy.

18. The computer-implemented method of claim 1, wherein the sample comprises DNA from a tumor.

19. The computer-implemented method of claim 17, wherein identifying informative variants involves filtering out variants above a given variant allele fraction (VAF) threshold in a matched germline reference sample.

20. The computer-implement method of claim 19, wherein the VAF threshold is between 50% and 99%.
